(19) United States
(12) Patent Application Publication
(10) Pub. No.: US 2024/0315249 A1
(43) Pub. Date: Sep. 26, 2024

(54) DISINFECTANT AND DISINFECTION METHOD FOR EFFECTIVELY KILLING ENDOSPORES

(71) Applicant: KUNMING QINGCHENG HEALTHCARE TECH LTD., Kunming (CN)

(72) Inventors: Lei ZHANG, Kunming (CN); Kewang LU, Kunming (CN); David LUK, Kunming (CN); Jie WEI, Kunming (CN); Xiaojun SHI, Kunming (CN)

(21) Appl. No.: 18/420,246

(22) Filed: Jan. 23, 2024

(30) Foreign Application Priority Data
Mar. 20, 2023 (CN) 202310268549.8

(51) Int. Cl. A01N 47/44; A01N 43/16; A01P 1/00

(52) U.S. Cl. CPC A01N 47/44; A01N 43/16; A01P 1/00

(57) ABSTRACT

The present disclosure discloses a disinfectant and a disinfection method for effectively killing endospores. The disinfectant includes a certain amount of polyhexamethylene biguanide as an active ingredient to kill endospores of Bacillus subtilis var. niger. The disinfectant can effectively kill endospores at room temperature and the atmospheric pressure. The disinfectant can be used for disinfection in environments including hospitals, crowded places, enclosed spaces, public transportation, and livestock and poultry breeding farms.

### CROSS-REFERENCE TO RELATED APPLICATIONS

This application claims priority to Chinese patent application No. 202310268549.8, filed on Mar. 20, 2023, the content of which is hereby incorporated by reference in its entirety.

### TECHNICAL FIELD

The present disclosure relates to the technical field of disinfectants, in particular to a disinfectant and a disinfection method for effectively killing endospores.

### BACKGROUND

Endospores, as metabolically dormant microorganisms, remain viability under various environments. They are the most stress-resistant organisms in the entire biosphere and exhibit outstanding resistance to heat, chemicals, and radiation. Distinguished from vegetative cells by their structure and chemical composition, endospores possess many characteristics distinct from those of vegetative cells. The main characteristic of endospores is their robust resistance, for example, to high temperature, ultraviolet radiation, dryness, ionizing radiation, and various toxic chemicals. Endospores of Clostridium botulinum take 5 to 9.5 hours to be killed in boiling water. The radiation resistance of endospores of Bacillus megaterium is 36 times stronger than that of E. coli cells. Endospores exhibit superior ability to maintain dormancy, and can remain viable for several years to decades under normal conditions. According to documented records, some endospores can even remain dormant for hundreds to thousands of years. Due to their stability, endospores can spread easily in environments they contaminate, especially in densely populated public places, such as hospitals, cinemas, shopping malls, airplanes, trains, ships, etc., necessitating frequent disinfection. In addition, with the economic development leading to increased import and export trade of goods, a significant volume of goods requires circulation. Thus, there is a need to disinfect during transit, especially for imported goods. For example, customs authorities necessitate effective disinfection procedures aiming to prevent infiltration of pathogens with the entry of goods causing epidemics.

Ethanol disinfectants commonly used by medical personnel cannot effectively kill endospores. For the disinfection of public places, the commonly used disinfectants are oxygen-containing disinfectants, chlorine-containing disinfectants, aldehyde disinfectants, or phenolic disinfectants. The oxygen-containing disinfectants include peroxyacetic acid, hydrogen peroxide, peroxyglutaric acid, chlorine dioxide, etc. However, peroxyacetic acid and peroxyglutaric acid are unstable and highly irritating, posing harm to the eyes and respiratory mucosa of human and animals upon prolonged use and causing severe damage to the environment. The chlorine-containing disinfectants refer to the disinfectants that can produce bactericidal hypochlorous acid in water. However, their metabolite, trichloromethane, is highly carcinogenic, and most of the chlorine-containing disinfectants are highly irritating. The aldehyde disinfectants, such as formaldehyde, glutaraldehyde, polyformaldehyde, etc., can produce free aldehyde radicals, reacting with microbial proteins and certain other components under appropriate conditions. However, the aldehyde disinfectants exhibit poor disinfection efficacy in the presence of organic contaminants. Moreover, formaldehyde and polyformaldehyde are highly irritating and carcinogenic, while glutaraldehyde is highly irritating to the eyes, skin, and mucous membranes and exhibits genotoxicity. The phenolic disinfectants, for example, halogenated phenol (such as chlorocresol), cresol (such as carbolic soap solution, known as Lysol), xylenol, bisphenols, complex phenols, etc., exhibit high carcinogenicity and accumulated toxicity, have a strong phenolic odor, and are ineffective against endospores. In addition, high-concentration hydrogen peroxide (25-30%) is capable of killing endospores but requires a long exposure time. Besides, high-concentration hydrogen peroxide is highly toxic, and can lead to poisoning by respiratory inhalation or mere skin contact. For example, exposure to 100 mg/kg of hydrogen peroxide in a short time will be life-threatening.

Endospore killing ability is one of the most important criterions in regulations for high-efficiency disinfectants or high-level disinfectants. In the health industry standard WS 628-2018 of the People's Republic of China, “Technical requirements for the hygiene and safety evaluation of disinfecting products,” Table B.1 “Items for testing disinfectants” outlines that the eradication test for endospores of Bacillus subtilis var. niger is a mandatory test for high-level disinfectants. In the national standard GB27949-2020 of the People's Republic of China, “General requirements for disinfectants of medical instruments”, Table 1 specifies that the kill log of endospores of Bacillus subtilis var. niger (ATCC9372) by high-level disinfectants should not be less than 3.00. Therefore, the endospore killing is an important indicator for evaluating high-efficacy disinfectants or high-level disinfectants.

China patent document CN113491709A discloses a multifunctional and efficient compound disinfectant and its preparation method. The disinfectant is mainly consisted of the following components: 0.01% to 2.0% of polyhexamethylene biguanide (PHMB), 0.05% to 0.2% of water-soluble quaternary ammonium salt, 0.1% to 2.0% of surfactant, 0.05% to 1.5% of thickener, 0.05% to 2.0% of moisturizer, 0.001% to 0.1% of flavor, 0 to 40.0% of medical ethanol, and the balance is pure water. This patent document discloses that the disinfectant demonstrates efficient bactericidal and long-term bacteriostatic effects, being capable of killing Escherichia coli, Staphylococcus aureus, Candida albicans, etc. within 1 minute. However, the efficacy against endospores is not disclosed. According to its formulation, polyhexamethylene biguanide and quaternary ammonium salt are the active components with bactericidal effects. Regarding polyhexamethylene biguanide, the patent in the background mentions that only 200 ppm of polyhexamethylene biguanide can achieve a bactericidal rate of 100% against Escherichia coli, Staphylococcus aureus, and Candida albicans, but it is not suitable for killing bacterial endospores. Besides, quaternary ammonium salt disinfectants are also not suitable for killing bacterial endospores or mycoplasma. Thus, this disinfectant cannot kill endospores, and thus cannot be considered as a high-efficacy disinfectant according to the common knowledge of those skilled in the art.

### SUMMARY

An object of the present disclosure is to provide a disinfectant, which is capable of killing endospores of Bacillus subtilis var. niger.

The present disclosure provides the following technical solutions.

A disinfectant capable of killing endospores includes polyhexamethylene biguanide as the active ingredient.

In some embodiments, a kill log of the disinfectant against endospores of Bacillus subtilis var. niger is greater than or equal to 3.00, achieving a killing rate thereof greater than 99.9%.

In some embodiments, the disinfectant is in a dosage form of gel or liquid.

The present disclosure reveals through experiments that in order to achieve high-efficacy disinfection effects, polyhexamethylene biguanide cannot coexist with a chelating agent, such as ethylenediaminetetraacetic acid or a salt thereof. The presence of the chelating agent can significantly decrease the endospore killing ability of polyhexamethylene biguanide. Thus, in some embodiments, the disinfectant does not include a chelating agent, such as ethylenediaminetetraacetic acid (EDTA) or a salt thereof, tetrasodium N,N-bis(carboxymethyl)-L-glutamate (GLDA), tetrasodium iminodisuccinate, trisodium N-(1-carboxylatoethyl)iminodiacetate (MGDA), diethylenetriaminepentaacetic acid (DPTA), a citrate salt, etc.

The present disclosure reveals through experiments that in order to achieve high-efficacy disinfection effects, polyhexamethylene biguanide cannot coexist with an oxidant. The presence of the oxidant can significantly decrease the endospore killing ability of polyhexamethylene biguanide. Thus, in some embodiments, the disinfectant does not include an oxidant, such as hydrogen peroxide, sodium hypochlorite, bromochlorodimethylhydantoin, etc.

In some embodiments, the disinfectant further includes a carrier or/and an acceptable excipient. The carrier is a solvent compatible with polyhexamethylene biguanide, such as water, propylene glycol, glycerol, polyethylene glycol, etc. The excipient includes at least one of a synergist, a thickener, etc. that is compatible with polyhexamethylene biguanide.

In some embodiments, the mass percentage of polyhexamethylene biguanide is greater than or equal to 0.2%, based upon the total mass of the disinfectant.

In some embodiments, the mass percentage of polyhexamethylene biguanide is in a range from 0.2% to 10%, from 0.2% to 9%, from 0.2% to 8%, from 0.2% to 7%, from 0.2% to 6%, from 0.2% to 5%, from 0.2% to 4.5%, from 0.2% to 4%, from 0.2% to 3.5%, from 0.2% to 3%, from 0.2% to 2.5%, from 0.2% to 2%, from 0.2% to 1.5%, from 0.2% to 1%, or from 0.2% to 0.5%, based upon the total mass of the disinfectant. In some embodiments, the mass percentage of polyhexamethylene biguanide is 0.2%, 0.3%, 0.4%, 0.5%, 1%, 1.5%, 2%, 2.5%, 3%, 3.5%, 4%, 4.5%, 5%, 6%, 7%, 8%, 9%, 10%, or within a range defined by any two of the above values.

In some embodiments, when the mass percentage of polyhexamethylene biguanide is less than 0.5%, such as in a range from 0.2% to 0.5%, although polyhexamethylene biguanide remains the ability to kill endospores of Bacillus subtilis var. niger, the required time period for the killing will be relatively prolonged. The present disclosure reveals that incorporating chitosan oligosaccharide as a synergist into the disinfectant can significantly enhance the endospore killing ability of polyhexamethylene biguanide. Therefore, in some embodiments, the mass percentage of polyhexamethylene biguanide is less than 0.5%, and the disinfectant further includes chitosan oligosaccharide. The combination of polyhexamethylene biguanide and chitosan oligosaccharide can enhance the efficacy of disinfection. In some embodiments, in the disinfectant including chitosan oligosaccharide, the mass percentage of chitosan oligosaccharide is greater than or equal to 0.2%.

In some embodiments, when the disinfectant does not include the synergist, the mass percentage of polyhexamethylene biguanide is greater than or equal to 0.5%, based upon the total mass of the disinfectant, so as to realize effective disinfection.

Another object of the present disclosure is to provide a method for killing endospores on an object or a surface, including applying the disinfectant to the object or the surface.

In some embodiments, the method includes maintaining the disinfectant on the object or the surface for a time period greater than or equal to 10 minutes, wherein the mass percentage of polyhexamethylene biguanide is greater than or equal to 0.5%, based upon the total mass of the disinfectant.

In some embodiments, the method includes maintaining the disinfectant on the object or the surface for a time period greater than or equal to 20 minutes, wherein the disinfectant further comprises chitosan oligosaccharide, the mass percentage of polyhexamethylene biguanide is in a range from 0.2% to 0.5%, and a mass percentage of chitosan oligosaccharide is greater than or equal to 0.2%, based upon the total mass of the disinfectant.

According to the present disclosure, when the mass percentage of polyhexamethylene biguanide is not less than 0.5% and the chelating agent is not present in the disinfectant, the disinfectant achieves a killing rate greater than 99.999% against endospores of Bacillus subtilis var. niger after 10 minutes of disinfection. When the mass percentage of polyhexamethylene biguanide is in a range from 0.2% to 0.5% and chitosan oligosaccharide is added as a synergist in the disinfectant, the disinfectant achieves a killing rate greater than 99.999% against endospores of Bacillus subtilis var. niger after 20 minutes of disinfection.

In view of the above, one or more embodiments of the present disclosure achieves one or more of the following beneficial effects.

1. The present disclosure reveals through experiments that polyhexamethylene biguanide at certain concentrations not only kills endospores, but also does so with high effectiveness. This challenges the prevailing misconception that polyhexamethylene biguanide, functioning as a cationic disinfectant, lacks the endospore killing ability in nature as mentioned in the background section of the patent document CN113491709A. In addition, the inventors of the present disclosure also found that among the common cationic disinfectants, polyhexamethylene biguanide might be the only one that possesses the ability to kill endospores effectively, which unveils extensive potential applications for polyhexamethylene biguanide in the field of disinfection.

2. The disinfectant of the embodiments of the present disclosure can effectively kill endospores at room temperature and one atmospheric pressure, and is non-toxic, non-irritating, and exhibit stable properties. Compared with the commonly used oxygen-containing disinfectants, chlorine-containing disinfectants, aldehyde disinfectants, or phenol disinfectants, the disinfectant of the embodiments of the present disclosure does not exhibits the shortcomings of other high efficiency disinfectants, thereby being expected to be a substitute of the existing disinfectants.

3. The present disclosure also reveals through experiments that the presence of the chelating agent can significantly decrease the endospore killing ability of polyhexamethylene biguanide, which provides a technical reference for the application and research involving polyhexamethylene biguanide.

4. The present disclosure also reveals through experiments that the presence of the oxidant can significantly decrease the endospore killing ability of polyhexamethylene biguanide, which provides a technical reference for the application and research involving polyhexamethylene biguanide.

5. The present disclosure also reveals through experiments that chitosan oligosaccharide is beneficial to enhancing the endospore killing ability of polyhexamethylene biguanide. The combination of polyhexamethylene biguanide and chitosan oligosaccharide can contribute to reduce the amount of polyhexamethylene biguanide needed.

6. The present disclosure has investigated the lowest effective concentration of polyhexamethylene biguanide and the minimum exposure time for polyhexamethylene biguanide to effectively kill endospores. These findings provide data reference for the research and development of high-efficacy disinfectant products.

7. The disinfectant of the embodiments of the present disclosure can be used for disinfection in environments including hospitals, crowded places, enclosed spaces, public transportation, livestock and poultry breeding farms, etc., and possesses green, safe, non-irritating, and stable properties, presenting significant market application prospects.

### DETAILED DESCRIPTION

In order to make the objectives, technical solutions, and advantages of the present disclosure more understandable and clearer, the present disclosure will be described in detail in combination with specific embodiments. It should be understood that the specific embodiments described herein are merely used to explain the present disclosure but are not intended to limit the present disclosure.

In the field of disinfectants, polyhexamethylene biguanide is a broad-spectrum antibacterial agent, and is commonly used as a cationic guanidine disinfectant. Polyhexamethylene biguanide exhibits effectiveness against Gram-positive bacteria, Gram-negative bacteria, fungi, and yeasts. The antibacterial mechanism is that the guanidine groups are highly active, and thus the polymer is positively charged and can be easily adsorbed by various bacteria and viruses, which are generally negatively charged, thereby preventing bacteria and viruses from dividing and reproducing, and causing bacteria and viruses to lose activity. This causes the cell membrane structure of bacteria and viruses to collapse, thereby forming transmembrane pores, rupturing the cell membrane, destroying the energy metabolism of the microorganisms, and causing bacteria and viruses to lose activity. The polymer forms a thin film to seal the breathing passages of microorganisms, thereby suffocating the microorganisms. In the medical field, polyhexamethylene biguanide is used for disinfection of contact lenses, eye drops, and surgical procedures. Additionally, it is also widely used in cosmetics, personal care products, textiles, the food industry, animal husbandry, swimming pools, etc. for sterilization, disinfection, and algae removal. Public information indicates that polyhexamethylene guanidine at a concentration of 0.02% achieves a kill rate of 100% against Escherichia coli, Staphylococcus aureus, Candida albicans, and gonococci. Polyhexamethylene guanidine at a concentration of 0.5% scores 3.0 points in an eye irritation test and scores 0 point in an acute skin irritation test, demonstrating its non-irritating property. Polyhexamethylene guanidine at a concentration of 1% can be administrated in a dosage greater than 5000 mg/kgBW, demonstrating its practical non-toxicity. Polyhexamethylene biguanide as a cationic guanidine disinfectant is conventionally classified as a low-efficacy disinfectant, and thus is not used for killing endospores. Based on this conception, currently there is no research record on polyhexamethylene biguanide in killing endospores. However, the present disclosure unexpectedly discovered through experiments that polyhexamethylene biguanide at a certain concentration can effectively kill endospores, thereby breaking the conventional bias. Based on this breakthrough, the present disclosure provides a disinfectant capable of effectively killing endospores, meeting various standards.

In the present disclosure, the term “kill log” refers to log reduction, and specifically refers to log₁₀ reduction of endospores before and after disinfection.

### Example 1

The disinfectant in this example is in a liquid form, and includes the following components: 0.5% by weight of polyhexamethylene biguanide, and deionized water as the balance.

Polyhexamethylene biguanide was introduced into deionized water according to the above mass percentage, then thoroughly mixed, and distributed into an appropriate container using a filling machine, thereby obtaining a finished product of the disinfectant.

The disinfectant in this example can further include one or more of the following components including a synergist and excipients according to the following mass percentages:
chitosan oligosaccharide: 0.1% to 1.0%;
chitosan: 1.0% to 10.0%; and
hydroxypropyl methylcellulose: 1.0% to 5.0%.

### Example 2

The disinfectant in this Example is in a gel form, and includes the following components: 0.3% by weight of polyhexamethylene biguanide, 5.0% by weight of chitosan, and deionized water as the balance.

The disinfectant was prepared as follows. The raw materials were taken according to the above mass percentages. Chitosan was first added to an appropriate amount of deionized water at a temperature of about 30° C. for fully swelling to obtain a gel A. Polyhexamethylene biguanide was then added to the remaining deionized water for fully dissolution, and uniformly mixed with the gel A. Finally, the mixture was distributed into an appropriate container using a filling machine, thereby obtaining a finished product of the disinfectant.

The disinfectant in this example can further include a synergist or/and an excipient according to the following mass percentages:
chitosan oligosaccharide: 0.1% to 1.0%; and
hydroxypropyl methylcellulose: 1.0% to 5.0%.

### Example 3

Fifteen formulations with their components and mass percentages (%) are listed in Table 2. The raw materials were taken in accordance with their mass percentages to prepare the disinfectants corresponding to the formulations.

TABLE 1
Components and mass percentages (%) of formulations

| Item | PHMB | CHX | BZK | D2 | D4 | BCDM | COS | CTS | HPMC |
|---|---|---|---|---|---|---|---|---|---|
| Formulation 1 | 4.5 | — | — | — | — | — | 10.0 | — | — |
| Formulation 2 | 4.5 | — | — | — | — | — | — | — | — |
| Formulation 3 | — | — | 0.2 | — | — | — | 10.0 | — | — |
| Formulation 4 | 4.5 | — | — | — | — | — | — | — | — |
| Formulation 5 | 1.0 | — | — | — | — | — | — | — | — |
| Formulation 6 | 1.0 | — | — | — | — | — | 10.0 | — | — |
| Formulation 7 | 1.0 | — | 0.4 | 0.4 | 0.6 | — | — | — | — |
| Formulation 8 | 1.0 | — | — | 1.0 | — | 0.02 | — | — | — |
| Formulation 9 | 0.5 | — | — | — | — | — | — | — | — |
| Formulation 10 | — | 0.5 | — | — | — | — | — | — | — |
| Formulation 11 | — | — | 0.5 | — | — | — | — | — | — |
| Formulation 12 | 0.2 | — | — | — | — | — | — | — | — |
| Formulation 13 | 0.2 | — | — | — | — | — | 0.2 | — | — |
| Formulation 14 | 0.3 | — | — | — | — | — | — | 5.0 | — |
| Formulation 15 | 0.1 | — | — | — | — | — | — | 5.0 | — |

In Table 2, PHMB is abbreviated for polyhexamethylene biguanide; CHX is abbreviated for chlorhexidine acetate; BZK is abbreviated for benzalkonium chloride; D2 is abbreviated for disodium ethylenediaminetetraacetate; D4 is abbreviated for tetrasodium ethylenediaminetetraacetate; BCDM is abbreviated for bromochlorodimethylhydantoin; COS is abbreviated for chitosan oligosaccharide; CTS is abbreviated for chitosan; and HPMC is abbreviated for hydroxypropyl methylcellulose. “-” means the formulation does not include that component.

To validate the effects of the present disclosure, the following verification experiments were performed.
I. Quantitative killing test for Bacillus subtilis var. niger endospore suspension
1. Main materials
1.1 Strain: Bacillus subtilis var. niger (ATCC9372).
1.2 Test samples of disinfectants: Thirteen samples prepared according to Formulations 1 to 13 in Table 1.
2. Test methods
2.1 A quantitative killing test for suspension was conducted according to section
2.1.1.7.4 of “Technical Standard for Disinfection” (the Ministry of Public Health of the People's Republic of China, 2002 edition), the content of which is hereby incorporated by reference in its entirety.
2.2 A neutralizer suspension quantitative identification test was conducted according to section 2.1.1.5.5 of “Technical Standard for Disinfection” (the Ministry of Public Health of the People's Republic of China, 2002 edition), the content of which is hereby incorporated by reference in its entirety.
3. The neutralizer used is Dey-Engley (D/E) neutralizing broth.
4. Test results

The test results are shown in Table 2.

TABLE 2
Test results for Bacillus subtilis var. niger endospores

| | | Colony- | | | | |
|---|---|---|---|---|---|---|
| | | forming unit | | Colony- | | |
| | | for positive | Log cfu/ml | forming unit | | |
| Sample and | Serial | control group | for positive | for test group | | Kill rate |
| exposure time | No. | (cfu/mL) | control group | (cfu/mL) | Kill Log | (%) |
| Formulation 1, | 1 | 1.5 × 10⁷ | 7.18 | <10 | >5.00 | >99.9999 |
| 60 minutes | 2 | 2.5 × 10⁷ | 7.40 | <10 | >5.00 | >99.9999 |
| | 3 | 1.7 × 10⁷ | 7.23 | <10 | >5.00 | >99.9999 |
| | Average | — | — | — | >5.00 | >99.9999 |
| Formulation 2, | 1 | 3.5 × 10⁷ | 7.54 | <10 | >5.00 | >99.9999 |
| 60 minutes | 2 | 4.0 × 10⁷ | 7.60 | <10 | >5.00 | >99.9999 |
| | 3 | 3.0 × 10⁷ | 7.48 | <10 | >5.00 | >99.9999 |
| | Average | — | — | — | >5.00 | >99.9999 |
| Formulation 3, | 1 | 3.5 × 10⁷ | 7.54 | 4.5 × 10⁶ | 0.89 | 87.1429 |
| 60 minutes | 2 | 4.0 × 10⁷ | 7.60 | 4.8 × 10⁶ | 0.92 | 88.0000 |
| | 3 | 3.0 × 10⁷ | 7.48 | 5.5 × 10⁶ | 0.74 | 81.6667 |
| | Average | — | — | — | 0.850 | 85.6032 |
| Formulation 4, | 1 | 1.5 × 10⁷ | 7.18 | <10 | >5.00 | >99.9999 |
| 20 minutes | 2 | 2.1 × 10⁷ | 7.32 | <10 | >5.00 | >99.9999 |
| | 3 | 1.7 × 10⁷ | 7.23 | <10 | >5.00 | >99.9999 |
| | Average | — | — | — | >5.00 | >99.9999 |
| Formulation 5, | 1 | 1.5 × 10⁷ | 7.18 | <10 | >5.00 | >99.9999 |
| 60 minutes | 2 | 2.1 × 10⁷ | 7.32 | <10 | >5.00 | >99.9999 |
| | 3 | 1.7 × 10⁷ | 7.23 | <10 | >5.00 | >99.9999 |
| | Average | — | — | — | >5.00 | >99.9999 |
| Formulation 5, | 1 | 3.4 × 10⁷ | 7.53 | <10 | >5.00 | >99.9999 |
| 20 minutes | 2 | 4.0 × 10⁷ | 7.60 | <10 | >5.00 | >99.9999 |
| | 3 | 2.9 × 10⁷ | 7.46 | <10 | >5.00 | >99.9999 |
| | Average | — | — | — | >5.00 | >99.9999 |
| Formulation 6, | 1 | 1.5 × 10⁷ | 7.18 | <10 | >5.00 | >99.9999 |
| 20 minutes | 2 | 2.1 × 10⁷ | 7.32 | <10 | >5.00 | >99.9999 |
| | 3 | 1.7 × 10⁷ | 7.23 | <10 | >5.00 | >99.9999 |
| | Average | — | — | — | >5.00 | >99.9999 |
| Formulation 7, | 1 | 2.1 × 10⁷ | — | 1.7 × 10⁷ | 0.09 | 19.05 |
| 20 minutes | 2 | 2.6 × 10⁷ | — | 2.2 × 10⁷ | 0.08 | 15.38 |
| | 3 | 3.3 × 10⁷ | — | 2.8 × 10⁷ | 0.07 | 15.15 |
| | Average | — | 7.43 | — | 0.080 | 16.53 |
| Formulation 8, | 1 | 2.0 × 10⁷ | — | 1.8 × 10⁷ | 0.04 | 10.00 |
| 20 minutes | 2 | 2.6 × 10⁷ | — | 2.4 × 10⁷ | 0.04 | 7.69 |
| | 3 | 3.0 × 10⁷ | — | 2.8 × 10⁷ | 0.03 | 6.67 |
| | Average | — | 7.40 | — | 0.037 | 8.12 |
| Formulation 9, | 1 | 3.4 × 10⁷ | 7.53 | <10 | >5.00 | >99.9999 |
| 20 minutes | 2 | 4.0 × 10⁷ | 7.60 | <10 | >5.00 | >99.9999 |
| | 3 | 2.9 × 10⁷ | 7.46 | <10 | >5.00 | >99.9999 |
| | Average | — | — | — | >5.00 | >99.9999 |
| Formulation 9, | 1 | 3.6 × 10⁷ | 7.56 | 2.6 × 10² | >5.00 | 99.9993 |
| 10 minutes | 2 | 4.5 × 10⁷ | 7.65 | 3.9 × 10² | >5.00 | 99.9991 |
| | 3 | 3.0 × 10⁷ | 7.48 | 2.3 × 10² | >5.00 | 99.9992 |
| | Average | — | — | — | >5.00 | 99.9992 |
| Formulation 9, | 1 | 3.6 × 10⁷ | 7.56 | 3.5 × 10⁶ | 1.01 | 90.2778 |
| 5 minutes | 2 | 4.5 × 10⁷ | 7.65 | 4.0 × 10⁶ | 1.05 | 91.1111 |
| | 3 | 3.0 × 10⁷ | 7.48 | 2.1 × 10⁶ | 1.15 | 93.0000 |
| | Average | — | — | — | 1.070 | 91.4630 |
| Formulation 10, | 1 | 3.4 × 10⁷ | 7.53 | 2.4 × 10⁷ | 0.15 | 29.4118 |
| 20 minutes | 2 | 4.0 × 10⁷ | 7.60 | 2.8 × 10⁷ | 0.15 | 30.0000 |
| | 3 | 2.9 × 10⁷ | 7.46 | 2.0 × 10⁷ | 0.16 | 31.0345 |
| | Average | — | — | — | 0.153 | 30.1488 |
| Formulation 11, | 1 | 3.6 × 10⁷ | 7.56 | 1.0 × 10⁶ | 1.56 | 97.2222 |
| 20 minutes | 2 | 4.5 × 10⁷ | 7.65 | 1.9 × 10⁶ | 1.37 | 95.7778 |
| | 3 | 3.0 × 10⁷ | 7.48 | 1.6 × 10⁶ | 1.27 | 94.6667 |
| | Average | — | — | — | 1.400 | 95.8889 |
| Formulation 12, | 1 | 3.6 × 10⁷ | 7.56 | 1.1 × 10⁵ | 2.51 | 99.6944 |
| 20 minutes | 2 | 4.5 × 10⁷ | 7.65 | 1.5 × 10⁵ | 2.48 | 99.6667 |
| | 3 | 3.0 × 10⁷ | 7.48 | 2.2 × 10⁵ | 2.13 | 99.2667 |
| | Average | — | — | — | 2.373 | 99.5426 |
| Formulation 12, | 1 | 3.6 × 10⁷ | 7.56 | 3.4 × 10⁶ | 1.02 | 90.5556 |
| 10 minutes | 2 | 4.5 × 10⁷ | 7.65 | 3.8 × 10⁶ | 1.07 | 91.5556 |
| | 3 | 3.0 × 10⁷ | 7.48 | 2.4 × 10⁶ | 1.10 | 92.0000 |
| | Average | — | — | — | 1.063 | 91.3704 |
| Formulation 13, | 1 | 3.6 × 10⁷ | 7.56 | 3.0 × 10² | >5.00 | 99.9992 |
| 20 minutes | 2 | 4.5 × 10⁷ | 7.65 | 4.4 × 10² | >5.00 | 99.9990 |
| | 3 | 3.0 × 10⁷ | 7.48 | 2.1 × 10² | >5.00 | 99.9993 |
| | Average | — | — | — | >5.00 | 99.9992 |

5. Test result analysis
5.1 From the test results of Formulations 1, 2, 4, 5, 6 and 9, it can be seen that when the mass percentage of polyhexamethylene biguanide was 0.5% and the exposure time was 20 minutes, 99.999% of the endospores of Bacillus subtilis var. niger can be effectively killed. However, when the mass percentage of polyhexamethylene biguanide fell below 0.5% in the absence of the synergist, the effectiveness against endospores of Bacillus subtilis var. niger diminished.
5.2 From the test results of Formulations 5, 7 and 8, it can be seen that when the mass percentage of polyhexamethylene biguanide was 1.0% and the exposure time was 20 minutes, the addition of the chelating agent (such as disodium ethylenediaminetetraacetate or tetrasodium ethylenediaminetetraacetate) and/or the oxidant (such as bromochlorodimethylhydantoin) can significantly decrease the endospore kill rate of polyhexamethylene biguanide. Formulation 5 achieves an endospore kill rate greater than 99.9999% after 20 minutes of exposure, while Formulations 7 and 8, under the same exposure time, only kill 16.53% and 8.12% of the endospores.
5.3 From the test results of Formulations 3 and 11, it can be seen that when the mass percentages of benzalkonium chloride were 0.2% and 0.5% respectively, the endospores of Bacillus subtilis var. niger cannot be effectively killed. At 0.2% of benzalkonium chloride and after 60 minutes of exposure, the endospore kill rate was 85.6032%, while at 0.5% of benzalkonium chloride and after 20 minutes of exposure, the endospore kill rate was 95.8889%.
5.4 From the test result of Formulation 10, it can be seen that when the mass percentage of chlorhexidine acetate was 0.5%, the endospores of Bacillus subtilis var. niger cannot be effectively killed. After 20 minutes of exposure, the endospore kill rate was 30.1488%.
5.5 From the test results of Formulations 12 and 13, it can be seen that when the mass percentage of polyhexamethylene biguanide was 0.2%, adding 0.2% of chitosan oligosaccharide can enhance the endospore killing ability of polyhexamethylene biguanide. 0.2% of polyhexamethylene biguanide alone achieved an endospore kill rate of 99.5426% after 20 minutes of exposure, while the combination of 0.2% of polyhexamethylene biguanide and 0.2% of chitosan oligosaccharide achieved an endospore kill rate of 99.9992% after 20 minutes of exposure.
5.6 From the test results of Formulation 12, it can be seen that when the mass percentage of polyhexamethylene biguanide was 0.2%, the endospore kill rates were 99.5426% (K=2.373) after 20 minutes of exposure and 91.3704% (K=1.063) after 10 minutes of exposure. Further reduction in polyhexamethylene biguanide concentration to 0.1% is expected to yield even lower endospore kill rate.
5.7 From the test results of Formulations 9, 10 and 12, it can be seen that 0.5% (Formulation 9) and 0.2% (Formulation 12) of polyhexamethylene biguanide respectively kill 99.9999% (K>5.00) and 99.5426% (K=2.373) of the endospores after 20 minutes of exposure, while 0.5% of chlorhexidine acetate (Formulation 10) achieves only an endospore killing rate of 30.1488% (K=0.153) after 20 minutes of exposure. This suggests that despite both are cationic guanidine disinfectants, polyhexamethylene biguanide can effectively kill endospores, while chlorhexidine acetate cannot effectively kill endospores, and thus is only a weaker disinfectant. Further, the combination of 0.1% of polyhexamethylene biguanide and 0.1% of chlorhexidine acetate exhibits significantly lower endospore killing ability compared to 0.2% of polyhexamethylene biguanide alone.
5.8 From the test results of Formulations 3, 9, 11 and 12, it can be seen that 0.5% (Formulation 9) and 0.2% (Formulation 12) of polyhexamethylene biguanide respectively kill 99.9999% (K>5.00) and 99.5426% (K=2.373) of the endospores after 20 minutes of exposure. In contrast, benzalkonium chloride as a cationic quaternary ammonium salt, when used in an amount of 0.5% (Formulation 11), achieves an endospore kill rate of 95.8889% (K=1.400) after 20 minutes of exposure; and when used in an amount of 0.2% (Formulation 3), it achieves an endospore kill rate of 85.6032% (K=0.850) after 60 minutes of exposure. This suggests that the common cationic quaternary ammonium salt as a disinfectant exhibits a weak efficacy, not effectively killing endospores, while polyhexamethylene biguanide can effectively kill endospores. Further, the combination of 0.1% of polyhexamethylene biguanide and 0.1% of cationic quaternary ammonium salt exhibits significantly lower endospore killing ability compared to 0.2% of polyhexamethylene biguanide alone.

The above descriptions are only embodiments of the present disclosure, and are not intended to limit the present disclosure. Any modifications, equivalent replacements and improvements made within the spirit and principles of the present disclosure should be included in the protection scope of the present disclosure.

What is claimed is:

1. A disinfectant capable of killing endospores, the disinfectant comprising:
polyhexamethylene biguanide as an active ingredient.

2. The disinfectant according to claim 1, wherein a kill log of the disinfectant against endospores of Bacillus subtilis var. niger is greater than or equal to 3.00.

3. The disinfectant according to claim 1, wherein the disinfectant is in a dosage form of gel or liquid.

4. The disinfectant according to claim 1, wherein the disinfectant does not comprise a chelating agent and/or an oxidant.

5. The disinfectant according to claim 1, wherein the disinfectant does not comprise at least one of ethylenediaminetetraacetic acid or a salt thereof, tetrasodium N,N-bis(carboxymethyl)-L-glutamate, tetrasodium iminodisuccinate, trisodium N-(1-carboxylatoethyl)iminodiacetate, diethylenetriaminepentaacetic acid, a citrate salt, hydrogen peroxide, sodium hypochlorite, or bromochlorodimethylhydantoin.

6. The disinfectant according to claim 1, further comprising a carrier or/and an acceptable excipient, wherein the carrier is a solvent compatible with polyhexamethylene biguanide, and the excipient comprises a synergist or a thickener that is compatible with polyhexamethylene biguanide.

7. The disinfectant according to claim 1, further comprising chitosan oligosaccharide.

8. The disinfectant according to claim 7, wherein a mass percentage of chitosan oligosaccharide is greater than or equal to 0.2%, based upon the total mass of the disinfectant.

9. The disinfectant according to claim 8, wherein the mass percentage of polyhexamethylene is greater than or equal to 0.2%, based upon the total mass of the disinfectant.

10. The disinfectant according to claim 8, wherein the mass percentage of polyhexamethylene biguanide is in a range from 0.2% to 0.5%, based upon the total mass of the disinfectant.

11. The disinfectant according to claim 1, wherein the mass percentage of polyhexamethylene biguanide is greater than or equal to 0.5%, based upon the total mass of the disinfectant.

12. The disinfectant according to claim 1, wherein the disinfectant does not comprise a water-soluble quaternary ammonium salt.

13. A method for killing endospores on an object or a surface, the method comprising:
applying the disinfectant according to claim 1 to the object or the surface.

14. The method according to claim 13, further comprising maintaining the disinfectant on the object or the surface for a time period greater than or equal to 10 minutes, wherein the mass percentage of polyhexamethylene biguanide is greater than or equal to 0.5%, based upon the total mass of the disinfectant.

15. The method according to claim 13, further comprising maintaining the disinfectant on the object or the surface for a time period greater than or equal to 20 minutes, wherein the disinfectant further comprises chitosan oligosaccharide, the mass percentage of polyhexamethylene biguanide is in a range from 0.2% to 0.5%, and a mass percentage of chitosan oligosaccharide is greater than or equal to 0.2%, based upon the total mass of the disinfectant.
